(19) United States
(12) Patent Application Publication
(10) Pub. No.: US 2005/0118247 A1
(43) Pub. Date: Jun. 2, 2005

(54) Method of making ketoprofen patch delivery system

(76) Inventors: Gunter Cordes, Langenfeld (DE); Ulrike Vollmer, Langenfeld (DE)

Correspondence Address: Clark G. Sullivan, Esq.;KING & SPALDING LLP, 45th Floor, 191 Peachtree Street, N.E., Atlanta, GA, 30303, US

(21) Appl. No.: 10/991,151

(22) Filed: Nov. 17, 2004

Related U.S. Application Data
(63) Continuation-in-part of application No. 10/332,221, filed on Apr. 24, 2003.

(30) Foreign Application Priority Data
Jul. 5, 2000 (DE) 100 32 537.8

(51) Int. Cl. A61K 9/70; A61K 9/24; A61K 31/192

(52) U.S. Cl. 424/449; 514/570; 427/2.31

(57) ABSTRACT

A controlled release ketoprofen patch for the topical application of ketoprofen is described, in addition to methods of treating inflammatory disorders and pain disorders by the administration of the controlled release ketoprofen patch.

### CROSS REFERENCE TO RELATED APPLICATIONS

The present application claims priority to U.S. patent application Ser. No. 10/332,221 filed Jul. 5, 2001, the contents of which are incorporated herein by reference.

### FIELD OF THE INVENTION

The invention provides transdermal patches for use in the controlled delivery of anti-inflammatory, analgesic, and/or antipyretic agents. In particular, a ketoprofen-containing transdermal patch useful in treating arthritis, inflammation, rheumatism, pain and sports-related muscle, tendon, cartilage and soft-tissue injuries is disclosed.

### DESCRIPTION OF RELATED ART

Inflammation is a widespread, non-specific response by a host to foreign antigens, often culminating in the rapid and efficient elimination of foreign substances. In general, there are four phases of inflammation—migration of leukocytes to the site of antigen localization; recognition of foreign antigens meditated by lymphocytes, macrophages, and complementary pathways; amplification of the inflammatory response; and macrophage, neutrophil, and lymphocyte participation in antigen destruction with ultimate foreign antigen removal by phagocytosis or similar cytotoxic mechanisms. Under normal circumstances, orderly progression of host defenses through these phases results in a well-controlled immune and inflammatory response that protects the host from the offending antigen. However, dysfunction of any of the host defense systems can result in damaged host tissue and the resultant clinically apparent diseases.

Non-steroidal anti-inflammatory drugs (NSAIDs) have been extensively used for decades in the treatment of inflammatory diseases such as arthritis and associated conditions. These compounds have been shown to exhibit anti-inflammatory, analgesic, and antipyretic activities, among others. While their mechanisms of action have not yet been fully established, it is known that their main mechanism of action is the inhibition of prostaglandin synthesis through the inhibition of cylo-oxygenase (COX).

A number of undesirable side effects have been attributed to NSAIDs such as gastrointestinal ulcerations and bleeding, as well as renal, skin and hypertension effects. [Rainsford, K. D., Am. J. Med., 107: pp. 27S-36S (1999); Perez Gutthann, S., et al., Epidemiology, 8: pp. 18-24 (1997); Moore, R. A., et al., British Med. J, 316: pp. 333-338 (1998)]. Additionally, NSAIDs can prolong bleeding time by effecting platelet function [Bergmann, J. F., et al., Eur. J. Clin. Pharmacol., 42: pp. 685-687 (1992)]. These problems are amplified by the fact that only a small percentage of orally administered NSAIDs actually reach the site of inflammation, thus necessitating relatively large doses of NSAIDs when ingested orally.

Due to these limitations on the oral use of NSAIDs, topical forms of NSAIDs have been developed to more precisely deliver the drugs to the site of inflammatory focus. These forms have proven particularly useful when inflammatory disorders are restricted to a superficial joint or to particular structures. A number of pharmacological studies have shown that topical forms of a variety of NSAIDs are useful in the treatment of inflammatory diseases and/or disorders such as myalgias, sprains, and tendonitis [Grahame, R., BJCP, 49: pp. 33-35 (1995); Heyneman, C. A., et al., Drugs, 60: pp. 555-574 (2000)].

Ketoprofen is an anti-inflammatory NSAID and is described chemically as 2-(3-benzoylphenyl)propionic acid. The compound is frequently used for relieving pain associated with musculoskeletal and joint diseases such as rheumatoid arthritis and osteoarthritis, as well as sports injuries such as sprains and tendonitis. Ketoprofen is typically administered orally in daily doses of from 100 to 300 mg [Kokki, H., et al., Eur. J. Clin. Pharmacol., 57: pp. 643-647 (2001)].

Ketoprofen is also marketed in various topical dosage forms, including gels and patches. One such gel, marketed by Aventis Pharma, Strasbourg, France, is Profenid® gel 2.5%. The product contains 25 mg of ketoprofen per gram of gel and has proven very popular in countries outside of the United States. The product suffers from a number of disadvantage including low bioavailability, declining plasma concentrations and rapid metabolization [Advenier, C., et al., Br. J. Pharmacol., 16: pp. 65-70 (1983)], as well as reported side effects such as photosensitization and contact eczemas [Veyrac, G., et al., Thérapie, 57: pp. 55-64 (2002)].

Ketoprofen patches and plasters (collectively “transdermal delivery systems” or “TDS”), have also been explored, including those described in U.S. Pat. No. 6,190,690 to Park, et al. and U.S. Pat. No. 5,730,999 to Lehmann, et al. In addition, a 70 cm² topical plaster containing 30 mg. of ketoprofen is marketed commercially outside the United States as Ketotop™ by Pacific Pharmaceuticals Co., Inc., Seoul, Korea. The Ketotop patch is administered two or more times daily.

Rolf, et al. [J. Rheumatology, 24: pp. 1595-1598 (1997)] reported the administration of a 30 mg, 70 cm² ketoprofen plaster u.i.d. for 5 days to thirty patients undergoing surgery for Achilles or patellar tendinopathy. Thirty patients in a separate group were treated orally with a single dose of 50 mg of ketoprofen prior to surgery. Daily administration of the patch was purportedly selected based upon an earlier pharmacokinetic study showing a plateau concentration of ketoprofen in plasma until the 24th hour of application. Concentrations of ketoprofen in plasma obtained after five days of patch administration were reported to be much lower than ketoprofen concentrations in plasma two hours after oral administration (a topical:oral route ratio of 0.0079).

In a subsequent paper, Rolf et al. [Rheumatology, 38: pp. 564-567 (1999)] explored the uptake of ketoprofen in synovial fluid, intra-articular tissues and plasma after topical applications of 30 mg, 70 cm² ketoprofen plasters u.i.d. for 5 days in thirty patients undergoing knee arthroscopy. The ketoprofen concentrations found in the different tissues after five days of patch administration were on average 18.7 ng/mL in plasma, 56.7 ng/g in synovial tissue, 569 ng/g in cartilage, and 12.8 ng/mL in synovial fluid. The tissue-to-plasma ratio in synovial tissue was thus reported to be approximately three.

In a paper presented in the Proceedings of the 2nd World Meeting of APGI/APV (Paris, May 25-28, 1998), Merten, et al. compared a 100 mg 90 cm² acrylic ketoprofen patch with 2.5% Gabrilen® ketoprofen gel and the Ketotop™ patch for in vitro release rates, release rates across hairless mouse skin, and plasma levels achieved when the patch is administered to humans. The patch reportedly achieving a modest AUC (area under the curve) (0-24 hr) (ng·hr/ml) of less than 500, and plasma concentrations of ketoprofen that reached a plateau about 10 hours after the patch was applied. Merten, et al. did not report the absolute rate of release of ketoprofen from their patch, the concentration of ketoprofen in their patch, the structure of the patch, or whether penetration enhancers were employed to increase the rate of ketoprofen release from the patch.

Despite the existence of many different types of delivery systems in the art, there exists a continuing need for alternate approaches to the delivery of ketoprofen to the target site of a patient in high concentrations over a prolonged period of time. What is needed especially is a ketoprofen patch that maximizes the ratio of ketoprofen at the site of inflammation in inflamed tissue relative to the concentration of ketoprofen in plasma, and delivers adequate amounts of ketoprofen into tissues beneath the stratum corneum to have an adequate therapeutic effect.

### SUMMARY OF THE INVENTION

The present invention provides a novel 100 mg, 90 cm² transdermal ketoprofen patch for the percutaneous administration of ketoprofen to localized inflammation beneath the skin surface. The patches are useful in the treatment of any inflammatory disorder—chronic, non-chronic and acute, but are particularly useful in the treatment of sports injuries such as tendonitis and sprains, due to the temporary nature of these injuries, and the ability of ketoprofen to penetrate to tissues damaged by these injuries. In the treatment of temporary pain, the patches are applied once per day, typically for up to about 14 days. When treating chronic pain, the patch will often be reapplied at a slower frequency, of from one to about 3-4 days, and it will be reapplied whenever pain emerges but potentially for prolonged periods of time. Regardless of the type of pain treated, and the frequency of reapplication, the patch preferably exhibits the following pharmacokinetic parameters—(i) a ketoprofen release rate in vivo of from about 10 to about 13 mg of ketoprofen per day, and (ii) a steadily increasing ketoprofen concentration in plasma during the initial days of treatment.

It has unexpectedly been discovered that these pharmacokinetic parameters result in ketoprofen concentrations in inflamed synovial tissues that are greater than about 4, 5 or 6 times as high as the concentration of ketoprofen observed in plasma. For example, the patches of the present invention produce a ratio of ketoprofen concentrations in synovial tissue versus plasma of up to about 6:1. In contrast, the Ketotop™ patch of the prior art releases only about 6 mg of ketoprofen per day and reaches a plateau ketoprofen concentration in plasma after only about 14 hours of the first administration, even when administered during successive days. Under these pharmacokinetics, the Ketotop™ patch was only able to achieve a ratio of ketoprofen concentrations in tissue vs. plasma of about 3:1 (i.e. ½ of the tissue:plasma ratio observed from the patches of the current invention).

Without wishing to be bound by any particular theory, it is believed that the higher rate of ketoprofen release achieved by the present patches synergistically drives greater amounts of ketoprofen into inflamed tissue relative to the ketoprofen that reaches the plasma. By driving greater proportions of ketoprofen into inflamed tissue relative to plasma, the patch maximizes the ketoprofen that has therapeutic effect at the site of inflammation, and minimize the ketoprofen that reaches the systemic circulation without first exerting a therapeutic effect. This result is surprising and could not have been predicted from the studies conducted in the prior art. This result is particularly surprising in view of the large surface area of the patch of the present invention—90 cm²—compared to the surface area of the Ketotop™ patch—i.e. 70 cm², because one would normally expect the tissue:plasma ratio to decrease as the surface area of the patch increased due to the increased distance between the ketoprofen and the inflamed tissue.

The steady increase in plasma ketoprofen concentrations observed for the patches of the current invention has also given rise to novel therapeutic regimens that combine the patch administrations of the present invention with the rapid onset of orally administered pain relief medications, especially non-prescription oral medications in the NSAID class such as ibuprofen, aspirin, and naproxen. In a preferred embodiment, a method for treating pain is provided in which an oral pain relief medication is administered first after a pain inducing episode, followed by daily administrations of the patch of the present invention. As the patch begins to have therapeutic effect, and its systemic concentration steadily increases, the use of the oral medication is gradually discontinued. The synergistic effect of this combination is demonstrated by placebo studies of clinical use.

As discussed above, the patches of the present invention have a surface area of 90 cm² and a ketoprofen content of 100 mg., and are intended for daily administration for the relief of chronic pain, or non-chronic temporary pain resulting, for example, from sports injuries such as tendonitis and sprains. In a preferred embodiment, the release rate of 10-13 mg/day from the 90 cm² 100 mg patch is obtained via supersaturation of the adhesive matrix in which the ketoprofen is solubilized, and the use of a free acid (which contributes to the supersaturated state). The patches of the present invention are also preferably characterized by one or more of the following physical attributes and performance characteristics:
The ketoprofen concentration in the patch matrix is optimally about 20 wt. %;
In non-chronic indications, the patch is preferably designed and/or sold with labeling indicia that instruct the user to reapply the patch daily for up to, and/or not to exceed, about 14 days;
The patch preferably is prepared without the addition of penetration enhancers;
The patch preferably yields an area under the curve (“AUC”) (0-24 hr) (ng·hr/ml) of from about 600 to about 3,500;
The patch preferably yields increasing plasma concentrations of ketoprofen greater than 4 ng/ml/day when applied for at least four consecutive days, when the patch is readministered every 1, 2, or 3 days;
When the patch is applied for eight consecutive days, it preferably yields concentrations of ketoprofen in plasma that exceed or equal about 100 ng/ml, whether reapplied once every one, two or three days; and
The release kinetics of the patch are preferably zero order or substantially zero-order.

Further objects, features and advantages of the present invention will be apparent to those skilled in the arts of pain management and topical delivery systems from consideration of the detailed description of preferred embodiments which follow.

### DESCRIPTION OF THE FIGURES

FIG. 1 is a top view of a transdermal skin patch of the present invention, showing the patch 1 and the release liner 2.

FIG. 2 is a cross-section of the transdermal skin patch of the present invention, having a release liner 2, a matrix adhesive layer containing the active drug 3, and cover layer 4.

FIG. 3 depicts a mean plasma concentration-time profile of ketoprofen (ng/ml) measured in volunteers after topical application of test formulation (patch) (dose=100 mg/day for 8 days), according to the method described in Comparative Example 1.

FIG. 4 is a reproduction of FIG. 2 from Merten (1998), and illustrates that the ketoprofen concentration in plasma reaches a plateau after about 14 hours when the transdermal patch disclosed in Merten (1998) is administered to humans once for a day.

### DEFINITIONS

The following definitions are provided in order to aid those skilled in the art in understanding the detailed description of the present invention.

The phrase, “substantially zero-order” as used herein means delivery of ketoprofen through the skin or mucosa at a rate which is approximately constant once steady state is attained. Plasma levels can vary up to about 10%, 20%, 30% or even 40%, from the mean in the plasma levels of ketoprofen at steady state (3-10 hours after administration), and still constitute “substantially zero order.”

The term “topical” or “topically” is used herein in its conventional meaning as referring to direct contact with skin on a human.

The term “treatment” or “treating” as used herein includes an approach for obtaining beneficial or desired results including but not limited to clinical results and the alleviation of symptoms.

The term “mean” when used in reference to patient response means the mathematical mean result achieved from 12 randomly selected patients, unless a different number of patients is specifically given.

### DETAILED DESCRIPTION OF THE INVENTION

Patch Structure

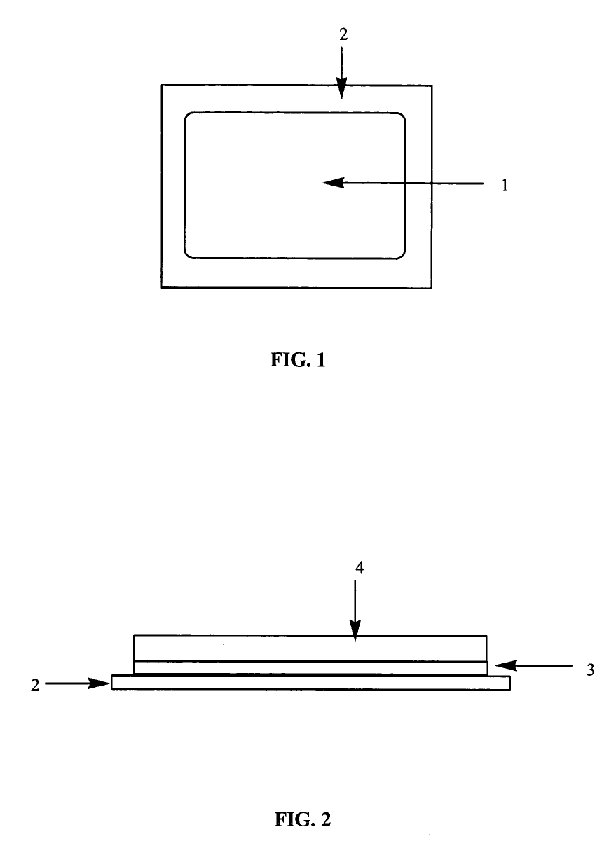

While the structure of patches of the present invention can vary, a preferred patch structure is depicted in FIGS. 1 and 2. As shown in FIGS. 1 and 2, the patch structure is preferably manufactured so that it comprises the patch 1 that is applied to the skin, and a removable protective layer/release liner 2 that is removed from the patch before application. The patch 1 preferably comprises an adhesive layer or matrix 3 and a non-reactive cover layer 4. The ketoprofen is integrated within the adhesive layer or matrix, and removal of the release liner 2 exposes the adhesive layer 3 which can then be applied to the skin near the site of inflammation. The patch structure including the patch 1 and removable protective layer 2 is preferably packaged in a foil package that is resistant to light and moisture. Other patch structures that the patches of the present invention can assume include those with additional layers, such as an adhesive layer between the drug matrix and release liner, or a primer between the drug matrix and the cover layer, as taught by Park et al. in U.S. Pat. No. 6,190,690.

The patch (i.e., the structure applied to the skin) is preferably rectangular in shape, with a surface area of from about 80 cm² to about 100 cm², preferably about 90 cm². The length:width ratio of the patch is preferably from about 1.3 to about 1.4, and optimally has surface dimensions of about 110×82 mm and a length:width ratio of about 1.34. The thickness of the matrix layer preferably is such that from about 40 g to about 70 g, from about 50 g to about 60 g, or from about 55 g or 56 g of the matrix layer 3 are present in each square meter of the patch (most preferably about 55.56 g/m²). The thickness of the matrix layer can vary from about 20 to about 500 micrometers, and is preferably from about 100 to about 350 micrometers, from about 200 to about 300, or from about 225 to about 275 micrometers in thickness when applied wet. In alternative embodiments, the thickness is greater than about 50 micrometers and less than about 400, 350, 325, 300, 275, or 250 micrometers in thickness when applied wet. It is surprising that one can attain the pharmacokinetic properties of the present patches based upon the foregoing dimensions, and it may be attributable, as discussed subsequently herein, to the supersaturated and/or free acid state of the ketoprofen.

A number of matrices for manufacturing patches are known in the art and are generally suitable for use in forming matrix layer 3, with the solvent based acrylic acid/acrylate, acrylate/vinyl acetate and acrylate/acrylic acid/vinyl acetate copolymers being particularly preferred. In a preferred embodiment, the monomeric blend comprises greater than about 50%, 60%, 70% 80% or 90% of acrylic acid, acrylate, and/or vinyl acetate monomers. A number of suitable acrylate copolymers are manufactured by National Starch & Chemical, BV, Zutphen, Netherlands under the DUROTAK trademark, and include DUROTAK™ 387-2825 (containing 2-ethylhexyl acrylate, vinyl acetate and acrylic acid), DUROTAK™ 387-2054 (containing butyl acrylate, 2-ethylhexylacrylate and acrylic acid), DUROTAK™ 87-2852 (containing 2-ethylhexyl methacrylate, methyl acrylate, vinyl acetate and acrylic acid), DUROTAK™ 387-2516 (containing 2-ethylhexyl acrylate, vinyl acetate, glycidyl methacrylate, and 2-hydroxymethyl acrylate), and DUROTAK™ 87-2070 (containing 2-ethylhexyl acrylate, acrylic acid, and glycidyl methacrylate). Preferred solvents for these copolymers include methanol, ethanol and 2-propanol, with 2-propanol being particularly preferred. Of these adhesives, DUROTAK™ 87-2852 solubilized in 2-propanol is most preferred due to its superior skin adhesion properties.

It has been found experimentally that this adhesive matrix has a balance of ionic charge, chain length and monomeric size that improves the ultimate performance of the patch. The —COOH function on the monomer may be an important contributor to this performance, and in various embodiments, the monomeric mix comprises greater than about 10%, 20% or 30% monomers with a —COOH function, and less than about 80%, 70% or 60% of monomers having such function. In one embodiment the formulations lack any significant amounts of a cross linker that would impact the adhesive properties or flexibility of the adhesive matrix.

The matrix layer also contains the active ingredient of the patch (i.e. the ketoprofen) solubilized in the matrix. Chemically, ketoprofen is 2-(3-benzoylphenyl) propionic acid, and has the following structure:

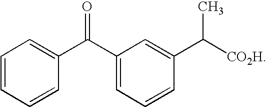

The ketoprofen used to make the patch can be in free acid, salt or ester form, though it is preferably supplied in the form of a free acid. Without wishing to be bound by theory, it is believed that the free acid form contributes to the supersaturated state of the drug, and the surprising uptake of ketoprofen in inflamed tissues. Moreover, while there are two enantiomers of ketoprofen (R and S) and a racemate that can be used to prepare the patches, the compound is preferably supplied as the racemate in accordance with the present invention.

The matrix layer preferably comprises from about 10 to about 30 wt. %, from about 15 to about 25 wt. %, from about 18 to about 22 wt. %, from about 20 to about 25 wt. %, or about 25 wt. % or about 20 wt. % ketoprofen, based on the solids content of the matrix layer. In a particularly preferred embodiment, the patch has an area of about 90 cm² and comprises about 100 mg of ketoprofen (corresponding to about 1.1 mg/cm²), preferably at a weight percentage of about 20%. In a preferred embodiment, the patch is packaged so that the ketoprofen remains solubilized in this concentrated state, without recrystallization, for at least 6 months, one year, eighteen months or two years.

The matrix is preferably supersaturated with ketoprofen, preferably in its free acid form. The ketoprofen is “supersaturated” in the sense that, when it is initially solubilized along with the matrix monomers in a suitable solvent medium, and as the solvent is progressively evaporated during the drying process, the ketoprofen is solubilized at a concentration greater than the established solubility level for ketoprofen in the solvent. In essence, the presence of the matrix monomers increases the solubility of the ketoprofen in the solvent. Without wishing to be bound by any theory, it is believed that this supersaturated state contributes to the excellent release characteristics of the patches of the present invention and causes greater than about 10 mg/day of ketoprofen to be released from the patch through the stratum corneum. In preferred embodiments, the ketoprofen concentration exceeds its known solubility in the solvent medium after about 50%, 70%, or 90% of the solvent has been evaporated. In a preferred embodiment, the supersaturation is achieved without the addition of any dissolution enhancing surfactants.

While penetration enhancers can be employed in the patches of the present invention, in a preferred embodiment the matrix layer lacks any meaningful amounts of such agents. Penetration enhancers are well known and are referred to in the art by terms such as skin-penetration enhancers, accelerants, adjuvants, and sorption promoters, all of which are referred to collectively herein as “penetration enhancers.” Agents within this class have diverse mechanisms of action, and include agents that improve the solubility and diffusibility of a drug within the multi-monomer polymeric matrix and those which improve percutaneous adsorption, for example, by changing the ability of the stratum corneum to retain moisture, softening the skin, improving the skin's permeability, acting as penetration assistants or hair-follicle openers or changing the state of the skin including the boundary layer.

Various pharmaceutically acceptable additives and excipients may also be incorporated into the matrix including tackifying agents, binders and rheological agents (i.e., thickeners). Other additives and excipients include diluents, stabilizers, fillers, clays, buffering agents, biocides, humectants, anti-irritants, antioxidants, preservatives, plasticizing agents, cross-linking agents, flavoring agents, colorants, pigments and the like.

The non-reactive cover layer 4 plays an important part in the wearability of the patch. Because the dermal system has to be applied to joints and other moving parts of the human body, a high degree of flexibility is necessary. It is also preferable that the cover layer 4 have good permeability to water vapor so as not to occlude the skin. Suitable materials for cover layer 4 include plastic films of polyethylene, vinyl acetate resins, ethylene/vinyl acetate copolymers, polyvinyl chloride, polyurethane, metal foils, woven fabrics, non-woven fabric, cloth and commercially available laminates. The backing material generally has a thickness in the range of from about 2 to about 1000 micrometers. A bidirectional elastic material (such as, for example, a woven polyester fabric), is particularly preferred.

Protective layer 2 is preferably a sheet-like material constructed of materials that are inert to the matrix layer, and that can be readily separated from the matrix layer. A particularly preferred material for protective layer 2 is a siliconized polyester foil, such as HOSTAPHAN™ RN 100 from Diafoil, Hoechst, Germany. The protective later is preferably at least about 36 micrometers thick, and most preferably about 100 micrometers thick, for ease of patient handling.

The matrix compositions according to the present invention can be prepared by first mixing appropriate amounts of the matrix material in volatile polar and/or non-polar organic liquids. An appropriate amount of ketoprofen is then added to the matrix material and the ingredients are thoroughly mixed. The ketoprofen is preferably added as a solution dissolved in methanol, ethanol, or 2-propanol. The mixture of the matrix composition is next formed into a film at ambient temperature, preferably by coating or casting at a controlled specified thickness onto a flexible sheet material, such as the protective layer 2, followed by evaporation of the volatile solvents at elevated temperatures (e.g., by passing through an oven). The matrix that has been coated or cast on the flexible sheet material is then laminated to another flexible sheet material, cover layer 4. Appropriate size and shape individual patches are then cut and packaged (e.g., pouched).

In a preferred embodiment, the invention provides a method of making a 90 cm² ketoprofen patch comprising a drug matrix, a cover layer, and about 100 mg of ketoprofen solubilized in the drug matrix, comprising:
a) preparing a solution that comprises a matrix precursor and ketoprofen;
b) spreading the solution onto a release liner;
c) drying the mixture to form a homogeneous laminate; and
d) lining the homogenous laminate with an elastic bidirectional cover layer.

However, it will be understood that the order of steps, the amount of the ingredients, and the amount and time of mixing may be important process variables which will depend on the specific polymers, active agents, solvents and/or co-solvents, enhancers and additives and excipients used in the composition. It will also be understood that additional layers, such as an adhesive layer between the drug matrix and release liner, or a primer between the drug matrix and the cover layer, could be integrated into the patch, as taught by Park et al. in U.S. Pat. No. 6,190,690. These factors can be adjusted by those skilled in the art, while keeping in mind the objects of achieving a solubilized active agent and providing a uniform product that will also give desirable results.

Patch Properties

The patch of the present invention preferably has a number of pharmacokinetic properties that make it especially useful in the applications of the present invention. Unless otherwise indicated, these pharmacokinetic properties are observed during any one or combination of days 1, 2, 3, 4, 5 or 6 when the patch is re-administered daily, and most preferably these properties are observed during the entire period of days 1-6.
an individual or mean release rate of from about 8 mg/day to about 15 mg/day, when applied once daily, preferably from about 10 mg/day to about 13 mg/day. This rate is measured by the amount of ketoprofen remaining in the patch after removal from the skin;
substantially zero order kinetics of release when the patch is readministered daily, over a 24, 48, or 72 hour period or longer;
treatment of inflamed tissue that is greater than 3, 5, 8, or 10 millimeters beneath the skin surface;
increasing plasma concentrations of ketoprofen for 2, 4, 6 or 8 consecutive days when the patch is readministered daily. The increase in ketoprofen concentration preferably exceeds or equals about 2, 4, 5 or 6 ng/ml/day of ketoprofen (individually or mean);
individual or mean concentrations of ketoprofen in plasma exceeding or equaling about 30, 40, 50, 75, 100 or 120 ng/ml after repeated daily administrations of said patch for 4, 6 or 8 continuous days; and
An “Area Under the Curve” (AUC) (0-24 hr) (ng·hr/ml) (mean or individual) of greater about 600, or ranging from about 600 to about 3,500, from about 800 to about 3,000, or from about 1,000 to about 2,500. This AUC can be observed on any or all of days 1, 2, 3, 4, 5 or 6 after daily administration of the patch of the present invention.

These properties are also preferably observed when the patch is applied continuously for a prolonged period of days, such as in chronic applications, but is reapplied at a frequency of every two, three or four days.

Therapeutic Application

The methods of the present invention are particularly useful in the treatment of temporary episodes of pain that last for one or more days but typically no more than about 14 days, such as pain that results from soft-tissue injuries and sports injury disorders such as tendonitis and joint sprains. The ketoprofen-containing transdermal delivery system (TDS) of the present invention is preferably administered once-daily for the treatment of temporary inflammatory conditions until such inflammatory condition subsides, typically for one or more days up to about fourteen consecutive days. In preferred embodiments, the patch is administered daily for from about 3 days to about 14 days, or from about 7 days to about 14 days, as well as any number of days between these two ranges. The patches of the present invention are particularly advantageous under these conditions of use because of the optimum adhesion that they exhibit with skin such that, even with close contact between the dermal system and the outer barrier of the skin for several days up to a maximum of one week, the system can be removed at any time without painful sensations or skin irritations. Because the patch has a substantially continuous rate of release over several days, the rate of reapplication can be lengthened to once every two, three or even four days.

In another embodiment, the patches of the present invention are used in the treatment of chronic pain. Because the patch continues to release ketoprofen at substantially the same rate for several days in a row, it is possible that the patch will remain on the user for two, three or even four days during inflammatory flare-ups associated with such chronic pain. Chronic inflammatory-related disorders treatable with the transdermal patch of the present invention include but are not limited to arthritis, including but not limited to rheumatoid arthritis, spondyloarthopathies, gouty arthritis, systemic lupus erythematosus, osteoarthritis and juvenile arthritis; asthma, bronchitis, menstrual cramps, tendonitis, bursitis, and skin related conditions such as psoriasis, eczema, burns and dermatitis; gastrointestinal conditions such as inflammatory bowel syndrome, Crohn's disease, gastritis, irritable bowel syndrome and ulcerative colitis; vascular diseases, migraine headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, hypersensitivity, conjunctivitis, gingivitis, swelling occurring after injury, myocardial ischemia, and the like; ankylosing spondylitis, cystic fibrosis, multiple sclerosis, acute pain (such as that from strains and sprains, and pain after surgery), primary dysmenorrheal, and peri-artiuclar disorders such as bursitis, and gout.

The site of inflammation treated by the present patches can be at various depths beneath the skin. Thus, for example, the inflammation tissue can be greater than about 3, 5, 7 or even 9 mm below the skin surface.

In a preferred embodiment, the patch is administered in combination with oral pain relief medication. The oral pain relief medication is administered before the patch is administered and shortly after the onset of pain, to provide initial pain relief. After the ketoprofen patch is administered and gradually begins to exert its therapeutic effect, the oral pain relief medication may be tapered off or discontinued entirely. Thus, in one embodiment the invention provides a method for treating non-chronic temporary pain in a patient comprising (a) administering an oral pain relief medication to said patient within about 12 hours of pain onset, and (b) subsequently administering daily, for up to about 14 consecutive days, the ketoprofen patch of the current invention. In a preferred embodiment, oral pain relief medication is discontinued within about 3, 2 or 1 days of the initiation of patch therapy.

The NSAIDs are particularly preferred oral pain medications, and include, for example, non-prescription oral medications in the NSAID class such as ibuprofen, aspirin, and naproxen. Other NSAIDs include the fenamic acid derivatives including flufenemic acid, mefenamic acid, meclofenamic acid, clonixeril, clonixin, flunixin, and diclofenac; benzenesulfonamides including rofecoxib and celecoxib; the indene derivatives including indomethacin, carprofen, etodolac, fendosal, indoprofen, prodolic acid, sermetacin, zidometacin, and zomeprirac, and the ibufenac derivatives including iflunisal, fenoprofen, alclofenac, amfenac, cliprofen, fenclofenac, fenclorac, fluprofen, ketoprofen, naproxol, genbufen, and ibufenac, as well as the pharmaceutically acceptable salts and esters thereof.

### EXAMPLES

The following examples are included to demonstrate preferred embodiments of the invention. It will be appreciated by those of skill in the art that the techniques disclosed in the examples represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Example 1

### Preparation of Ketoprofen Patch

To 30.83 g of a 36% (w/w) solution of an acrylate adhesive (Durotak 87-2852, National Starch & Chemical B.V., NL-Zutphen) was added a solution of 2.78 g of 2-(3-benzophenyl)propionic acid in 5.6 g of 2-propanol. The solution was homogenised by stirring for one hour and was then spread out, using a doctor blade, onto a siliconised, 100 um-thick polyester film (FL 2000 100u 1-S, Rexam Release B.V., NL-Apeldoorn) in a wet-layer thickness of 260 um. After drying (1 h at 40° C. and 50 min at 80° C.), the clear and homogenous laminate was lined with a woven bidirectional polyester (M02/97, white, K. O. Braun, D-Wolfstein) without stretching. The completed patch is 90 cm² in size, has a matrix weight of about 55.6 g/m², contains 100 mg. of 2-(3-benzophenyl)propionic acid, exhibits an adhesive strength [N/25 mm] of 6.8±0.6, and exhibits a separating force [N/25 mm] of 0.137±0.012.

### Example 2

### Analysis of Ketoprofen Concentrations in Inflamed Tissues

An open, repeated dose study was undertaken, examining a number of subjects for a period of 6 days. One transdermal patch, prepared substantially according to Example 1 and containing a 100 mg dose of ketoprofen, was applied on the knee region or on the tunnel carpale region once a day, in the morning, for six consecutive days. Each of the first five patches were retained on the subjects for 24 hours, while the last patch was retained on the subject for 6 hours, and was removed just prior to arthroscopy or endoscopy.

Subjects were operated on under spinal anaesthesia (meniscus' arthroscopy) or local anesthesia (endoscopic carpal tunnel release) after the six consecutive days of application of the ketoprofen patch of the present invention. Prior to operation, the skin was disinfected appropriately according to standard preoperative routines. During the knee arthroscopy, biopsies were taken from the synovial tissue of the medial compartment and from the anterior fat pad (Hoffa). During endoscopic carpal tunnel release a biopsy of the ulnar bursa was taken when entering the carpal tunnel with the endoscope. Samples were immediately frozen at −20° C. following excision. Venous blood was drawn by direct venipuncture on day 6, before removing the last patch during insertion of the i.v. line. The withdrawn blood was introduced into heparinized test tubes, kept on ice, and rapidly centrifuged. The resulting plasma was separated, divided into two aliquots, and transferred into two polypropylene tubes that were then stoppered such that they were airtight, and then the tubes were frozen at −20° C.

Biological samples were transferred frozen to the I.P.A.S. Analytical Unit. Plasma was stored at −20° C., while tissues were stored at −80° C. up until the time of the assay. Ketoprofen was assayed in plasma by a validated LC-MS-MS method. Ketoprofen in tissues was assayed against a calibration curve in plasma; QC samples correspondent to an animal matrix were correctly quantified with a plasma curve, as well as using a cross-validation for tissue determination. In the case of tissues, concentrations <1 ng/mL were appreciated according to a semiquantitative method.

Individual plasma ketoprofen concentrations in the patients are shown in Table 1. The mean value of plasma concentration was 52.8 ng/mL, ranging from 12.9 ng/mL to 112.0 ng/mL. Table 1 also lists the individual ketoprofen concentrations in all tissues examined. During knee meniscus arthroscopy, biotic samples of anterior fat pad and synovial tissues were drawn (subjects 1, 3, 5, 7 and 10), while during endoscopic carpal tunnel operation the tendon sheath was sampled (subjects 2, 4, 6, 8 and 9), as described previously.

TABLE 1
Ketoprofen concentrations in plasma, intra-articular adipose tissue, synovial
tissue, and tendon sheath after 6 patch applications (100 mg u.i.d.)

| | | Knee | | | | Wrist | |
|---|---|---|---|---|---|---|---|
| | | Anterior | | Synovial | | Tendon | |
| | | Fat Pad | | Tissue | | Sheath | |
| | Plasma | Tiss. | Tissue/ | Tiss. | Tissue/ | Tiss. | Tissue/ |
| | Concentration | conc. | Plasma | conc. | Plasma | conc. | Plasma |
| Subjects | (ng/mL) | (ng/g) | ratio | (ng/g) | ratio | (ng/g) | ratio |
| 1 | 80.1 | 15.4 | 0.19 | 300.0 | 3.75 | | |
| 2 | 38.5 | | | | | 18,003.5 | 467.62 |
| 3 | 54.5 | 41.9 | 0.77 | 129.1 | 2.37 | | |
| 4 | 39.5 | | | | | 19.134.1 | 484.41 |
| 5 | 12.9 | 7.7 | 0.60 | 20.0 | 1.55 | | |
| 6 | 81.4 | | | | | 17,813.2 | 218.84 |
| 7 | 21.8 | 7.2 | 0.33 | 315.3 | 14.46 | | |
| 8 | 112.0 | | | | | 32,578.2 | 290.88 |
| 9 | 42.0 | | | | | 13,004.2 | 309.62 |
| 10 | 45.2 | 67.1 | 1.48 | 430.5 | 9.52 | | |
| n | 10 | 5 | 5 | 5 | 5 | 5 | 5 |
| Mean | 52.8 | 27.9 | 0.67 | 239.0 | 6.33 | 20,106.6 | 354.27 |
| SD | 30.1 | 26.1 | 0.45 | 163.0 | 4.93 | 7,358.9 | 104.06 |
| CV % | 57.0 | 93.5 | 66.91 | 68.2 | 77.86 | 36.6 | 29.37 |
| Min | 12.9 | 7.2 | 0.19 | 20.0 | 1.55 | 13,004.2 | 218.84 |
| Max | 112 | 67.1 | 1.48 | 430.5 | 14.46 | 32,578.2 | 484.41 |

### Comparative Example 1

### Plasma Concentrations After Topical Administration of Ketoprofen Patch and Ketoprofen Gel

In this example, a ketoprofen patch substantially as described in example 1 was applied once daily for six consecutive days to 12 different patients, and the patients were evaluated daily for plasma ketoprofen concentrations. As a comparison, 50 mg of Profenid ketoprofen gel (2.5%) was applied twice daily for six consecutive days to 12 different patients, and the patients were evaluated daily for plasma ketoprofen concentrations. The results are presented in Table 2 below.

TABLE 2

| | | | Mean | |
|---|---|---|---|---|
| | Mean | | Ketoprofen | |
| | Ketoprofen | Range of | Plasma | Range of |
| | Plasma | Ketoprofen | Concen- | Ketoprofen |
| | Concentration | Concentrations | tration | Concentrations |
| | (ng/ml) − TDS | (ng/ml)-TDS | (ng/ml) − | (ng/ml)- |
| | formulation | formulation | Profenid | Profenid |
| Day 2 | 15.18 | 5.33-35.88 | 21.85 | n.e.-62.70 |
| Day 4 | 24.16 | 5.01-75.13 | 20.95 | 4.79-48.18 |
| Day 6 | 41.81 | 14.02-99.58 | 19.1 | 4.01-99.57 |

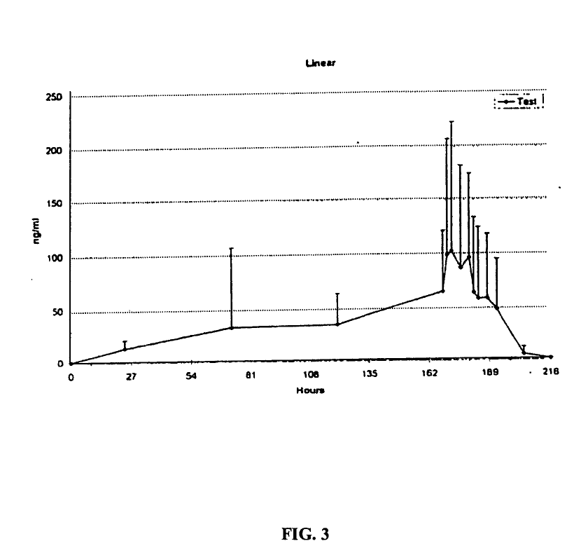

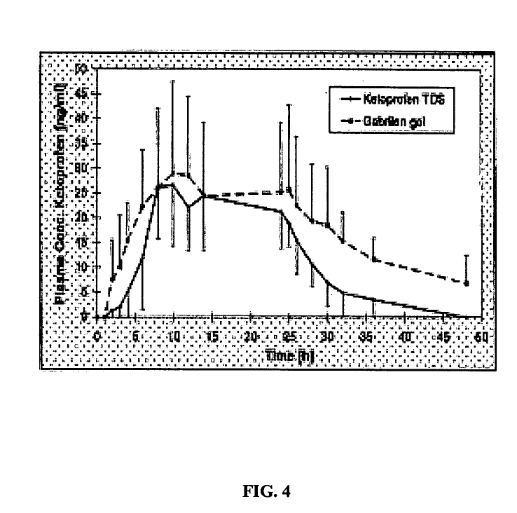

As can be seen, daily patch administrations result in a continuously increasing concentration of ketoprofen in plasma, with the average ketoprofen concentration increasing at about 7 ng/ml/day. In contrast, no increase in plasma ketoprofen concentrations was observed after the second day for the gel applications. A mean plasma concentration-time profile of ketoprofen (ng/ml) measured in all volunteers after topical application of test formulation (patch) (dose=100 mg/day for 8 days) is depicted in FIG. 3.

### Comparative Example 2

### Merten (1998)

The following example is taken from Merten (1998), and demonstrates the plateau effect of a comparative ketoprofen patch having a 100 mg loading of ketoprofen. The precise construction of the patch, the rate of ketoprofen release from the patch, the concentration of ketoprofen in the patch matrix, and the presence or absence of penetration enhancers is not reported. After administration of a single patch over a 24 hour period, Merten (1998) reports a mean Area Under the Curve (AUC) (0-24 hr) (ng hr/ml) of 437.1. In contrast, the mean AUC (0-24 hr.) (ng hr./ml) for the patch of example 1 was found experimentally to be 1847.3 based upon the administration of the patch to 24 healthy volunteers, which is nearly a four-fold increase.

FIG. 4 is a reproduction of FIG. 2 from Merten (1998), and illustrates that the ketoprofen concentration in plasma reaches a plateau after about 10-15 hours when a single transdermal patch disclosed in Merten (1998) is administered to humans. This plateau effect is consistent with the plateau effect reported in Rolf (1999) that reportedly occurred 14 hours after administration of a 30 mg. ketoprofen patch, but stands in contrast to the continuous increase in plasma concentration observed when the ketoprofen patch of example 1 is reapplied to the skin daily for eight continuous days, as illustrated in FIG. 3.

### Example 3

### Comparative Example—Rolf (1997) and Rolf (1999)

The following comparative example reproduces data reported in Rolf (1997) and Rolf (1999). Concentrations reported are in ng/g for tissue, and ng/ml for fluid. The data was obtained after 5 days of daily administrations of a 30 mg, 70 cm² ketoprofen plaster patch. The data reported was obtained immediately after removing the fifth patch.

TABLE 3
Ketoprofen Concentration Data from Rolf (1997) and Rolf (1999)

| TISSUE | Rolf (1997) | Rolf (1999) |
|---|---|---|
| Synovial Tissue | — | 56.7 |
| Meniscus | — | 349.3 |
| Cartilage | — | 568.9 |
| Plasma | 17.87 | 18.7 |
| Synovial Fluid | — | 12.8 |
| Skin | 332,337.7 | — |
| Fat | 2453.9 | — |
| Tendon Sheath | 5026.3 | — |
| Tendon | 952.8 | — |

Table 4 contains a comparison of tissue to plasma concentration ratios as reported in Example 2, Rolf (1997) and Rolf (1999). As can be seen, the patch of the present invention achieves a synovial tissue/plasma ratio that is about twice as large as the synovial tissue/plasma ratio reported in Rolf (1999), and a tendon sheath/plasma ratio that is about half again as large as the tendon sheath/plasma ratio reported in Rolf (1997).

TABLE 4

| | Example 2 | Rolf (1997) | Rolf (1999) |
|---|---|---|---|
| Synovial tissue/plasma | 6 | — | 3 |
| Tendon sheath/plasma | 354 | 273 | — |

All of the compositions, methods, and/or processes disclosed and claimed herein can be made and executed without undue experimentation in light of the present disclosure. While the compositions and methods of this invention have been described in terms of preferred embodiments, it will be apparent to those of skill in the art that variations may be applied to the compositions, methods, and/or processes and in the steps or in the sequence of steps of the methods described herein without departing from the concept and scope of the invention. For example, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1) A method of making a 90 cm² ketoprofen patch comprising a drug matrix, a cover layer, and about 100 mg. of ketoprofen solubilized in said drug matrix, comprising:
a) preparing a solution that comprises a matrix precursor and ketoprofen;
b) spreading said solution onto a release liner;
c) drying said mixture to form a homogeneous laminate; and
d) lining said homogenous laminate with an elastic bidirectional cover layer.

2) The method of claim 1 wherein said matrix precursor comprises one or more acrylic monomers.

3) The method of claim 1 wherein said ketoprofen is the solubilized residue of a free acid.

4) The method of claim 1 wherein said solution comprises methanol, ethanol or 2-propanol.

5) The method of claim 1 wherein said matrix precursor and said ketoprofen are present in a weight ratio of from about 85:15 to about 75:25.

6) The method of claim 1 wherein said solution is prepared by mixing separate solutions of matrix precursor and ketoprofen.

7) The method of claim 1 wherein said solution is spread onto said release liner at a thickness of from about 100 um to about 300 um.

8) The method of claim 1, wherein said ketoprofen is supersaturated in said solution.

9) The method of claim 1 wherein said drug matrix is an adhesive matrix.

10) The method of claim 1, wherein said drug matrix comprises means for achieving supersaturation of said ketroprofen in said matrix.

11) The method of claim 1 wherein said patch achieves one or more of the following in vivo pharmacokinetic properties:
a) a ketoprofen release rate of from about 8 mg to about 15 mg of ketoprofen per day;
b) an increase in plasma ketoprofen concentrations of greater than about 2 ng/ml/day when the patch is readministered daily for 4 days or more;
c) a ketoprofen plasma concentration exceeding 50 ng/ml when the patch is readministered daily for 8 consecutive days; and/or
d) an AUC (0-24 hr) (ng·hr./ml) ranging from about 600 to about 3,500.

12) The method of claim 1 wherein said ketoprofen is present in said drug matrix at a concentration of from about 18 wt. % to about 22 wt. %.

13) The method of claim 1 wherein said ketoprofen is present in said drug matrix at a concentration of about 20 wt. %.

14) The method of claim 1 wherein said patch achieves increases in ketoprofen concentrations of greater than about 4 ng/ml/day when the patch is re-administered daily for 4 days or more.

15) The method of claim 1 wherein said patch achieves a ketoprofen plasma concentration exceeding 75 ng/ml when the patch is readministered daily for 8 consecutive days.

16) The method of claim 1 wherein said patch achieves an AUC (0-24 hr) (ng·hr./ml) ranging from about 800 to about 3,000.

17) The method of claim 1 wherein said patch has a rectangular shape and a length:width ratio of from about 1.30 to about 1.40.

18) The method of claim 1 wherein said drug matrix has an area weight of from about 40 g/m² to about 70 g/m².

19) The method of claim 1 wherein said drug matrix has an area weight of from about 50 g/m² to about 60 g/m².

20) The method of claim 1 wherein said patch substantially lacks a penetration enhancer.

21) The method of claim 1 wherein said cover layer comprises a bidirectional elastic material.
